(19) United States
(12) Patent Application Publication
(10) Pub. No.: US 2017/0000889 A1
(43) Pub. Date: Jan. 5, 2017

(54) STABLE BROMFENAC OPHTHALMIC SOLUTION

(71) Applicant: SENTISS PHARMA PRIVATE LIMITED, New Delhi (IN)

(72) Inventors: Mandar V. Shah, New Delhi (IN); Mukesh Tiwari, New Delhi (IN); Deepak Bahri, New Delhi (IN)

(21) Appl. No.: 15/100,015

(22) PCT Filed: Dec. 11, 2014
(86) PCT No.: PCT/IB2014/066784

§ 371 Date: May 27, 2016

(30) Foreign Application Priority Data
Dec. 12, 2013 (IN) 3626/DEL/2013

(51) Int. Cl. A61K 47/26; A61K 9/00; A61K 9/08; A61K 31/196

(52) U.S. Cl. CPC A61K 47/26; A61K 31/196; A61K 9/0048; A61K 9/08

(57) ABSTRACT

The present invention provides a stable, aqueous solution comprising a non-steroidal anti-inflammatory drug (NSAID) such as bromfenac or a pharmacologically acceptable salt or a hydrate thereof and a solubilizer. The present invention provides methods to reduce the level of degradation of bromfenac with the addition of pharmaceutically acceptable solubilizers other than an alkyl aryl polyether alcohol type polymer such as tyloxapol, and/or a polyethylene glycol fatty acid ester such as polyethylene glycol monostearate. Also provided are stabilized compositions, and methods of their manufacture and use, e.g., for the treatment of ocular inflammation and pain after cataract surgery.

### FIELD OF THE INVENTION

The present invention is directed to stable, aqueous solution comprising a non-steroidal anti-inflammatory drug (NSAID) such as bromfenac or a pharmacologically acceptable salt or a hydrate thereof and/or pharmaceutically acceptable excipients which does not comprises an alkyl aryl polyether alcohol type polymer such as tyloxapol, or a polyethylene glycol fatty acid ester such as polyethylene glycol monostearate for the treatment of ocular inflammation and pain after cataract surgery. More particularly the present invention is directed to reduce the level of degradation of bromfenac with the addition of pharmaceutically acceptable solubilizers other than an alkyl aryl polyether alcohol type polymer such as tyloxapol, or a polyethylene glycol fatty acid ester such as polyethylene glycol monostearate.

### BACKGROUND

Bromfenac (chemical name 2-amino-3-(4-bromobenzoyl)phenylacetic acid), a non-steroidal anti-inflammatory agent, is disclosed in JP-A-23052/1977 and its corresponding U.S. Pat. No. 4,045,576, as well as in Japanese patent no. 2683676 corresponding to U.S. Pat. No. 4,910,225. The sodium salt of bromfenac has been used ophthamologically, e.g., in the form of eye drops. Bromfenac is effective against inflammatory diseases (e.g. blepharitis, conjunctivitis, scleritis, postoperative inflammation) of the extraocular segment or the anterior ocular segment in the field of ophthalmology, and in particular, its efficacy for treating uveitis is equal to nonsteroidal anti-inflammatory agents which have previously been used in the field of ophthalmology, and its sodium salt has been practically used in the form of eye drops. The eye drop as mentioned above is designed to stabilize 2-amino-3-(4-bromobenzoyl)phenylacetic acid by means of addition of a water-soluble polymer (e.g. polyvinylpyrrolidone, polyvinyl alcohol, etc.) and a sulfite (e.g. sodium sulfite, potassium sulfite, etc.) (Japanese patent No. 2,683,676 and its corresponding U.S. Pat. No. 4,910,225).

In addition, as an eye drop other than the above-mentioned one, Japanese patent No. 2,954,356 (corresponding to U.S. Pat. Nos. 5,603,929 and 5,653,972) discloses a stable ophthalmic composition which comprises incorporating an antibacterial quaternary ammonium polymer and boric acid into an acidic ophthalmic agent. The acidic agent described therein includes, for example, 2-amino-3-(4-bromobenzoyl)phenylacetic acid.

U.S. Pat. No. 8,129,431, US2012115957, US2013090384, US2007287749 and WO2013055856 disclose an aqueous liquid preparation containing 2-amino-3-(4-bromobenzoyl)phenylacetic acid or its pharmacologically acceptable salt or a hydrate thereof, an alkyl aryl polyether alcohol type polymer such as tyloxapol, or a polyethylene glycol fatty acid ester such as polyethylene glycol monostearate.

The present invention provides a stable, aqueous solution comprising bromfenac or its pharmacologically acceptable salt or hydrate thereof and/or pharmaceutically acceptable excipients wherein the solution does not comprise an alkyl aryl polyether alcohol type polymer such as tyloxapol, and/or a polyethylene glycol fatty acid ester such as polyethylene glycol monostearate.

Additionally the stable, aqueous solution of the present invention does not comprise antioxidants such as sulfite(s) but not limited to sodium sulfite, potassium sulfite and the like. Sodium sulfite is irritating to the eyes. Symptoms of irritation may include redness, itching or tearing as disclosed and mentioned in various companies Material Safety Data Sheet (MSDS) such as Santa Cruz Biotechnology, Inc.; LabChem, Inc., New Jersey department of health and senior services and the like.

The inventors of the present invention with expenditure of intellectual effort and careful experimentation have prepared a stable, aqueous solution comprising a non-steroidal anti-inflammatory drug (NSAID) and/or pharmaceutically acceptable excipients for the treatment of ocular inflammation and pain after cataract surgery wherein more specifically, the present invention is directed to reduce the level of degradation of bromfenac with the addition of pharmaceutically acceptable solubilizers such as an polyoxyethylene sorbitan fatty acid esters such as polysorbate 80 or a non-ionic solubilizer such as polyoxyl-15-hydroxystearate.

There is a need for a method for treating ocular inflammation and pain after cataract surgery wherein the method comprises a once a day or twice a day topical application to the eye of the patient in need of a stable, aqueous solution comprising bromfenac or a pharmacologically acceptable salt or a hydrate thereof and/or pharmaceutically acceptable excipients.

### OBJECT OF THE INVENTION

The main object of the present invention is to develop a stable, aqueous solution comprising bromfenac or a pharmacologically acceptable salt or a hydrate thereof, for the treatment of ocular inflammation and pain after cataract surgery.

Yet another object of the present invention is to develop a stable solution within a pH range giving no irritation to eyes.

Yet another object of the present invention is to remove the anti-oxidants/stabilizer, sodium sulfite from the formulation of the control formulation (PROLENSA) as sodium sulfite is a known irritant to the eye. In the process, inventors of this formulation stabilized it more than that of the control formulation, with respect to the formation of Impurity B.

Yet another objective of the present invention is to remove the anti-oxidants/stabilizer, sodium sulfite from the formulation of the control formulation (PROLENSA) as sodium sulfite is a known irritant to the eye. In the process, inventors of this formulation stabilized it more than that of the control formulation, with respect to the formation of Impurity B.

Yet another object of the present invention is directed to reduce the level of degradation of bromfenac with the addition of pharmaceutically acceptable solubilizers other than an alkyl aryl polyether alcohol type polymer such as tyloxapol, or a polyethylene glycol fatty acid ester such as polyethylene glycol monostearate.

Yet another object of the present invention is to develop a method to prepare a stable, aqueous solution comprising bromfenac or its pharmacologically acceptable salt or hydrate thereof and/or pharmaceutically acceptable excipients wherein the invention does not comprise an alkyl aryl polyether alcohol type polymer such as tyloxapol, and/or a polyethylene glycol fatty acid ester such as polyethylene glycol monostearate.

Yet another object of the present invention provides a method for treating ocular inflammation and pain after cataract surgery wherein the method comprises a once a day or twice a day topical application to the eye of the patient in need of a stable, aqueous solution comprising bromfenac or a pharmacologically acceptable salt or a hydrate thereof and/or pharmaceutically acceptable excipients.

### SUMMARY OF THE INVENTION

The present invention is directed to a stable, aqueous solution comprising bromfenac or a pharmacologically acceptable salt or a hydrate thereof, for the treatment of ocular inflammation and pain after cataract surgery wherein the solution is stable within a pH range giving no irritation to eyes.

The present invention is directed to stable, aqueous solution which does not comprise an alkyl aryl polyether alcohol type polymer such as tyloxapol, and/or a polyethylene glycol fatty acid ester such as polyethylene glycol monostearate for the treatment of ocular inflammation and pain after cataract surgery.

More particularly the present invention is directed to reduce the level of degradation of bromfenac with the addition of pharmaceutically acceptable solubilizers other than an alkyl aryl polyether alcohol type polymer such as tyloxapol, or a polyethylene glycol fatty acid ester such as polyethylene glycol monostearate.

More specifically the invention provides a method for stabilizing an aqueous solution of bromfenac or a pharmacologically acceptable salt or a hydrate thereof by adding a solubilizer such as a polyoxyethylene sorbitan fatty acid esters such as polysorbate 80 or a non-ionic solubilizer such as polyoxyl-15-hydroxystearate to an aqueous solution of bromfenac, wherein the aqueous solution becomes stable within acceptable pH range giving no irritation to eyes, and unexpectedly reduces the level of degradation of bromfenac.

Additionally the solution of the present invention does not comprise an antioxidant such as sulfite(s) preferably sodium sulfite and potassium sulfite and can be formulated both as multi-dose as well as unit-dose composition.

The present invention provides a method to prepare a stable, aqueous solution comprising bromfenac or its pharmacologically acceptable salt or hydrate thereof and/or pharmaceutically acceptable excipients wherein the invention does not comprise an alkyl aryl polyether alcohol type polymer such as tyloxapol, and/or a polyethylene glycol fatty acid ester such as polyethylene glycol monostearate.

The present invention provides a method for treating ocular inflammation and pain after cataract surgery wherein the method comprises a once a day or twice a day topical application to the eye of the patient in need of a stable, aqueous solution comprising bromfenac or a pharmacologically acceptable salt or a hydrate thereof and/or pharmaceutically acceptable excipients wherein more specifically, the present invention does not comprise an alkyl aryl polyether alcohol type polymer such as tyloxapol, and/or a polyethylene glycol fatty acid ester such as polyethylene glycol monostearate.

### BRIEF DESCRIPTION OF THE DRAWINGS

FIG. I shows the comparative leukocyte counts between Group I (Negative control), Group II (Present invention formulation—036A) & Group III (Innovator product—PROLENSA™).

FIG. II shows the comparative vasodilation reduction between Group I (Negative control), Group II (Present. Invention formulation—036A) & Group III (Innovator product—PROLENSA™).

FIG. III shows the levels of “Impurity B” in Control formulation—PROLENSA™ and present invention formulation (batch 35B & 35C) at accelerated stability condition (40° C./NMT 25% RH).

### DETAILED DESCRIPTION OF THE INVENTION

As used herein, an “antioxidant” is a substance that when present in a mixture containing an oxidizable substrate biological molecule significantly delays or prevents oxidation of the substrate biological molecule. In the context of this invention, antioxidants include, but are not limited to, antioxidants that are irritating to the eyes. Such antioxidants include sodium sulfite, potassium sulfite, sodium metabisulfite, sodium thiosulfate, acetylcysteine, butylated hydroxyanisole, butylated hydroxytoluene and the like, and mixtures thereof.

As used herein, the term “° C.” wherever appears is an abbreviation for “Degree Celsius”.

As used herein, the term “BKC” wherever appears is an abbreviation for “benzalkonium chloride”.

As used herein, the term “Control” wherever appears is a marketed product “PROLENSA™”.

As used herein, the “bromfenac sodium” wherever appears is an abbreviation for “bromfenac sodium salt or hydrate thereof, wherein the hydrate is at least one selected from a 1/2 hydrate, 1 hydrate and 3/2 hydrate”.

As used herein, the “NMT” wherever appears is an abbreviation for “not more than”.

Unless indicated otherwise, all ingredient amounts are presented in units of % weight/volume (% w/v).

As used herein, the “CGYS” wherever appears is an abbreviation for “Clear greenish yellow solution”.

One embodiment of the present invention, is to develop a stable, aqueous solution comprising bromfenac or a pharmacologically acceptable salt thereof or a hydrate thereof, for the treatment of ocular inflammation and pain after cataract surgery wherein which is stable within a pH range giving no irritation to eyes.

Other embodiment of the invention is to provide a method for stabilizing an aqueous solution of bromfenac or a pharmacologically acceptable salt thereof or a hydrate thereof wherein more specifically, the present invention does not comprise an alkyl aryl polyether alcohol type polymer such as tyloxapol, and/or a polyethylene glycol fatty acid ester such as polyethylene glycol monostearate.

In yet other embodiment of the present invention, citrate buffer is used instead of borate buffer but when benzalkonium chloride (BKC) was added in the citrate formulation it causes haziness of the formulation. It is an object of the present invention to stabilize the aqueous solution, in which, when a preservative such as benzalkonium chloride is incorporated therein, does not result in haziness, preferably for the shelf-life of the solution.

The inventors of the present invention extensively studied how to overcome the haziness occurred during the addition of BKC. Surprisingly it was found that surfactants such as polyoxyethylene sorbitan fatty acid esters such as polysorbate 80 or a non-ionic solubilizer such as polyoxyl-15-hydroxystearate (Kolliphor® HS 15 or Solutol® HS 15) remove the haziness. In addition several other surfactants such as Kolliphor® TPGS (Vitamin E Polyethylene Glycol Succinate USP/NF, Tocophersolan (CAS number 9002-96-4)) and Brij™ C20 also removed haziness. However, different surfactants affected stability of bromfenac in a different manner and surprisingly it was found that polysorbate 80 or polyoxyl-15-hydroxystearate (Kolliphor® HS 15 or Solutol® HS 15) has the least amount of degradation of the present solution.

So the inventors of the present invention after careful experimentation have identified surfactants that clarify the solution (remove or avoid haziness) and also reduce the level of degradation of bromfenac.

In another embodiment, the invention provides a method for stabilizing an aqueous solution of bromfenac or a pharmacologically acceptable salt or a hydrate thereof by adding a solubilizer such as a polyoxyethylene sorbitan fatty acid ester such as polysorbate 80 or a non-ionic solubilizer such as polyoxyl-15-hydroxystearate to an aqueous solution of bromfenac, the aqueous solution becomes stable within a pH range giving no irritation to eyes, and unexpectedly reduces the level of degradation of bromfenac.

As a result of various studies, the inventors of the present invention have found that, by adding one or more solubilizers to an aqueous solution of bromfenac or a pharmacologically acceptable salt or a hydrate thereof, the aqueous solution becomes stable within a pH range giving no irritation to eyes, and unexpectedly reduces the level of degradation of bromfenac. Some preferred solubilizers in context to the present invention includes but not limited to, for example, a polyoxyethylene sorbitan fatty acid ester such as polysorbate 80 or a non-ionic solubilizer such as polyoxyl-15-hydroxystearate such as Kolliphor® HS 15.

One embodiment of the present invention, is to develop a stable, aqueous solution comprising bromfenac or a pharmacologically acceptable salt thereof or a hydrate thereof and a solubilizer such as an polyoxyethylene sorbitan fatty acid esters such as polysorbate 80 and/or pharmaceutically acceptable excipients, for the treatment of ocular inflammation and pain after cataract surgery wherein which is stable within a pH range giving no irritation to eyes.

Another embodiment of the present invention, is to develop a stable, aqueous solution comprising bromfenac or a pharmacologically acceptable salt thereof or a hydrate thereof and a non-ionic solubilizer such as polyoxyl-15-hydroxystearate and/or pharmaceutically acceptable excipients, for the treatment of ocular inflammation and pain after cataract surgery wherein which is stable within a pH range giving no irritation to eyes.

Yet another embodiment of the invention is to provide a method for stabilizing an aqueous solution of bromfenac or a pharmacologically acceptable salt or a hydrate thereof by adding a solubilizer, preferably polsorbate 80 or Polyoxyl-15-Hydroxystearate.

In a preferred embodiment, the present invention provides a method to prepare a stable, aqueous solution comprising bromfenac or its pharmacologically acceptable salt or hydrate thereof and/or pharmaceutically acceptable excipients wherein the invention does not comprise an alkyl aryl polyether alcohol type polymer such as tyloxapol and a polyethylene glycol fatty acid ester such as polyethylene glycol monostearate.

In another preferred embodiment, the present invention provides a method to prepare a stable, aqueous solution comprising bromfenac or its pharmacologically acceptable salt or a hydrate thereof and/or pharmaceutically acceptable excipients and a solubilizer. Preferred solubilizers in context to the present invention includes but not limited to, for example, a polyoxyethylene sorbitan fatty acid ester such as polysorbate 80 or a non-ionic solubilizer such as polyoxyl-15-hydroxystearate such as Kolliphor® HS 15.

In yet another preferred embodiment of the present invention, the stable, aqueous solution of the present invention is also devoid of antioxidants, preferably devoid of sulfites, more preferably devoid of sodium sulfite, potassium sulfite and the like which causes irritation to the eyes wherein symptoms of irritation may include redness, itching or tearing. So, the absence of sodium sulfite adds to the benefit for better patient compliance.

In another preferred embodiment of the present invention, citrate buffer is used, preferably instead of borate buffer.

Furthermore in another preferred embodiment, the present invention provides a method for treating ocular inflammation and pain after cataract surgery wherein the method comprises application to the eye of the patient in need of a stable, aqueous solution comprising bromfenac and a solubilizer such as an polyoxyethylene sorbitan fatty acid esters such as polysorbate 80 or a non-ionic solubilizer such as polyoxyl-15-hydroxystearate. Application is preferably once a day, but may be more than once per day, e.g., two times a day.

Furthermore in another preferred embodiment, the present invention provides a method of using the inventive compositions.

Furthermore in yet another preferred embodiment, the present invention provides a clear, isotonic, sterile bromfenac ophthalmic aqueous solution, useful for the treatment of ocular inflammation and pain after cataract surgery wherein the solution is contained in a unit dose kit form and is applied once a day to each eye.

In another embodiment, aqueous solution of the present invention may also be packaged in a single-use container and/or multi-use container.

In another preferred embodiment the stable, ophthalmic compositions can also be prepared as one of the embodiments of the present invention to make the composition pharmaceutically acceptable for used as a single or multi-unit dose.

Any concentration of NSAID that is effective can be used, and can be determined by one of ordinary skill in the art using the present disclosure as guidance. When the NSAID comprises bromfenac, bromfenac is preferably present in a concentration of 0.01% w/v-0.1% w/v (based on bromfenac free base). Some preferred concentrations include 0.02, 0.04, 0.05, 0.06, 0.07, 0.08, and 0.09% w/v bromfenac and ranges formed from these values. These bromfenac concentrations may be used with the excipients and amounts thereof listed in Table 1.

In an embodiment, the present invention provides a stable, aqueous solution of bromfenac or a pharmacologically acceptable salt or a hydrate thereof, wherein the pharmacologically acceptable salt of bromfenac includes, but not limited to, for example, sodium salt; potassium salt; calcium salt and magnesium salt, wherein sodium salt is especially preferable.

In another embodiment, the present invention provides a stable, aqueous solution of bromfenac or a pharmacologically acceptable salt or a hydrate thereof, wherein the hydrate is at least one selected from a 1/2 hydrate, 1 hydrate and 3/2 hydrate.

The aqueous solution of the present invention is stable at 50° C. for four weeks wherein the stability result clearly demonstrates that the assay of bromfenac and related substance are well within specification ranges. The bromfenac content in the formulation of present invention (containing polysorbate 80 as a solubilizer) is 101.5%, compared to 99.3% of the label in control formulation and the bromfenac content in the formulation of present invention (containing Polyoxyl-15-Hydroxystearate as a solubilizer) is 100.7%, compared to 99.3% of the label in control formulation respectively at 50° C. for four (4) weeks. Also the related substance (total impurities) in the formulation of present invention (containing polysorbate 80 as a solubilizer) is 0.30%, compared to 0.33% of the label in control formulation and the related substance (total impurities) in the formulation of present invention (containing Polyoxyl-15-Hydroxystearate as a solubilizer) is 0.32%, compared to 0.33% of the label in control formulation respectively at 50° C. for four (4) weeks. Hence, one can conclude that the formulation of present invention is unexpectedly as stable as that of the control at pH 7.8, without the need for, or use of, antioxidants such as sulfite(s) such as sodium sulfite, potassium sulfite, and the like, and/or with the addition of pharmaceutically acceptable solubilizers such as either an polyoxyethylene sorbitan fatty acid esters such as polysorbate 80 or a non-ionic solubilizer such as polyoxyl-15-hydroxystearate.

“Stable” refers to low bromfenac degradation and/or low formation of degradation products (or “related substance”) after aging. Stable formulations preferably include those in which bromfenac assay is greater than or equal to 97%, more preferably 98%, 99% or 99.5% after aging. Stable formulations preferably include those in which the related substance assay is less than 3%, more preferably less than 2.5, 2.0, and 1.0% after aging. Aging may be accelerated or non-accelerated, preferably accelerated. Accelerated aging preferably comprises storage at 50° C. for two (2) weeks, preferably in a closed container in the dark. As is known in the art, “related substances” preferably include substances regulated by regulatory authorities, e.g., the U.S. FDA. Related substances preferably include Impurity A as well as the highest unknown impurity, and more preferably includes all bromfenac degradation products. These impurities can be determined and assayed by one of ordinary skill in the art. As is known in the art, Impurity A is 7-(4-bromobenzoyl)-1,3-dihydro-2H-indol-2-one.

In one of the preferred embodiments, excipients used are ophthalmically acceptable which includes, without limitation, solubilizers, buffering agents, chelating agents, tonicity agents, permeation enhancers, surfactants, pH adjusting agents, preservatives and the like.

In another preferred embodiment, compositions of the present invention may include one or more buffering agent. Preferred buffering agents include, but are not limited to, acetate buffers, citrate buffers, phosphate buffers, sodium dihydrogen phosphate dihydrate, dibasic sodium phosphate heptahydrate, monobasic sodium phosphate, citric acid, citric acid monohydrate or ε-aminocaproic acid and the like. Preferred buffers include citrate buffers.

In another preferred embodiment, compositions of the present invention may comprise one or more chelating agents. Preferred chelating agents include, but are not limited to, disodium edetate or ethylenediamine tetraacetic acid (“EDTA”), diammonium EDTA, dipotassium EDTA, calcium di sodium EDTA, hydroxyethylethylenediaminetriacetic acid (“HEDTA”), ethylenediaminetetraacetic acid, mono(triethanolamine) salt (“TEA-EDTA”), tetrasodium EDTA, tripotassium EDTA, trisodium phosphate, diammonium citrate, galactaric acid, galacturonic acid, gluconic acid, glucuronic acid, cyclodextrin, potassium citrate, the potassium salt of ethylenediamine-tetra (methylene phosphonic acid) (“EDTMP”), sodium citrate, sodium EDTMP, and the like.

When a preservative is used, any ophthalmically acceptable preservative may be used. When used, preferred preservatives include, but are not limited to, one or more of quaternary ammonium salts (e.g. benzalkonium chloride, benzethonium chloride, etc.), chlorhexidine gluconate, benzethonium chloride, benzododecinium bromide, quaternary ammonium compounds such as but not limited to benzethonium chloride, methylbenzethonium chloride, cetalkonium chloride, cetylpyridinium chloride, cetrimonium, cetrimide, dofanium chloride, tetraethylammonium bromide, didecyldimethylammonium chloride, domiphen bromide and the like; Polyquaternium-1 (Polyquad®), 1phenyl ethanol, phenyl propanol, phenyl mercuric acetate, phenyl mercuric nitrate, phenyl mercuric borate, chlorhexidine acetate or gluconate, chlorocresol, benzoic acid, benzyl alcohol, butylparaben, propylparaben, methylparaben, chlorobutanol, phenoxyethanol, sodium methyl paraben, sodiumpropyl paraben, thimerosal, and mixtures thereof. Any effective amount may be used, preferably from 0.005% to 0.5% (w/w).

In yet another preferred embodiment of the present invention, tonicity adjusting agents may be added. Preferred tonicity agents include, without limitation, glycerin, sorbitol, sodium hydroxide, sodium chloride, potassium chloride, mannitol, dextrose, and propylene glycol, as well as combinations thereof, or any other suitable ophthalmically acceptable tonicity adjusting agents.

In other preferred embodiments of the present invention, vehicles can also be used in the ophthalmic compositions of the present embodiments. These vehicles include, but are not limited to, methylcellulose, hydroxypropyl methylcellulose, poloxamers, carboxymethyl cellulose, hydroxyethyl cellulose, polyethylene glycol, hyaluronic acid, polygalacturonic acid, xyloglucan, carbopol, polycarbophil, gellan gum, physiological saline solution, water, purified water, and combinations thereof.

Aqueous compositions may comprise any suitable amount of water. Preferred aqueous compositions comprise more than 90% water by weight, more preferably more than 95% water, or more than 98% water by weight.

The present invention is preferably devoid of any kind of antioxidants such as sodium sulfite, potassium sulfite, sodium metabisulfite, sodium thiosulfate, acetylcysteine, butylated hydroxyanisole, butylated hydroxytoluene and the like and mixtures thereof which causes irritation to the eyes.

In another preferred embodiment, surfactants, when used, may be selected from the group consisted of, but are not limited to sodium lauryl sulfate, docusate sodium, polyoxyalkyl ethers, polyoxylalkyl phenyl ethers, polyoxyl 40 hydrogenated castor oil (Cremophor RH 40), polyoxy hydrogenated castor oil, polyoxy sorbitan esters, sorbitan esters, polysorbates, an polyoxyethylene sorbitan fatty acid esters such as Polysorbate 20; Polysorbate 21; Polysorbate 40; Polysorbate 60; Polysorbate 61; Polysorbate 65; Polysorbate 80; Polysorbate 81; Polysorbate 85; Polysorbate 120, polyoxyl 35 castor oil, sorbitan monolaureates, poloxamer and non-ionic surfactant such as Kolliphor® TPGS (Vitamin E Polyethylene Glycol Succinate USP/NF, Tocophersolan (CAS number 9002-96-4)); non-ionic solubilizer such as Kolliphor® HS 15 or Solutol® HS 15 (polyoxyl-15-hydroxystearate) and mixtures thereof.

Any ophthamically acceptable pH adjusting agent may be used. Preferred pH adjusting agents include hydrochloric acid, sodium hydroxide, phosphoric acid, acetic acid and the like.

The excipients used in the present invention are preferably selected to be non-toxic and have no substantial detrimental effect (preferably, in the amount used) on the present ophthalmic compositions, on the use of the compositions or on the human or animal to which the ophthalmic compositions are to be administered.

In preferred embodiments, the present invention provides ophthalmic compositions in the form of aqueous liquids, solutions, emulsion, dispersion, suspension, reverse emulsion and microemulsion, nanoemulsion, nano reservoir system, in-situ gel drops, nanoparticulate system, liposomal drops, bioadhesive gel drops, drops and the like.

In another preferred embodiment, the present invention provides ophthalmic compositions for topical ophthalmic delivery comprising administering said composition in the eyes. Other preferred embodiments include otic and/or nasal formulations for administration to the ear and/or nose of a human or animal.

In a preferred embodiment, the stable, solution is an aqueous solution having a pH value within the range of from about 6.5 to about 9, especially from about 6.8 to about 8.0 and preferably from about 7.0 to about 7.8. In another preferred embodiment, the inventive composition has osmolality in range of at least about 250 mOsmol/kg and/or less than or equal to about 350 mOsmol/kg.

In an especially preferred embodiment, the osmolality or tonicity of the carrier component substantially corresponds to the tonicity of the fluids of the eye, in particular the human eye. The pH of the aqueous solution of the present invention is closer to ocular or lacrimal fluid as compared to the marketed product.

In a yet further preferred embodiment, the present invention provides a process of preparing a stable, aqueous solution comprising bromfenac and/or pharmaceutically acceptable excipients.

Still further, the present invention may also preferably be presented as a kit comprising a stable, aqueous solution comprising bromfenac and/or pharmaceutically acceptable excipients, the aqueous solution being contained within a container prepared from a pharmaceutically acceptable packaging material.

Any pharmaceutically acceptable packaging material may be use, preferably packaging material that is suitable for containing ophthalmic aqueous solution, more preferably bromfenac ophthalmic aqueous solution. Pharmaceutically acceptable packaging materials include but are not limited to low density polyethylene (“LDPE”), high density polyethylene (“HDPE”), polypropylene, polystyrene, polycarbonate, polyesters (such as polyethylene terephthalate and polyethylene naphthalate), nylon, polyvinyl chloride), poly(vinylidine chloride), poly(tetrafluoroethylene) and other materials known to those of ordinary skill in the art. Flexible bottles prepared from, or comprising, LDPE, HDPE or polypropylene are particularly preferred.

Preferred containers include bottles, preferably a dropper (e.g., a bottle or ampule suitable for dropwise application of the composition), more preferably, a single-use bottle or dropper. The containers are preferably sterilized, preferably prior to filling. Any suitable method can be used to sterilize the containers, and can be determined by the person of ordinary skill in the art. Some preferred methods include exposure to gamma irradiation and/or exposure to ethylene oxide gas.

The aqueous solution is preferably sterile. An article comprising the aqueous solution filled in a container is preferably sterile, preferably at the time the container is filled. The aqueous solution is preferably filled into sterile multi-use or single-use containers.

The present invention provides a method for treating ocular inflammation and pain after cataract surgery wherein the method comprises a once a day or twice a day topical application to the eye of the patient in need of a stable, aqueous solution comprising bromfenac or a pharmacologically acceptable salt or a hydrate thereof and/or pharmaceutically acceptable excipients and a solubilizer (preferably an polyoxyethylene sorbitan fatty acid esters such as polysorbate 80 or a non-ionic solubilizer such as polyoxyl-15-hydroxystearate).

In some of the main embodiments, the present invention provides the following:
1. A stable pharmaceutical composition comprising bromfenac, or a pharmaceutically acceptable salt or hydrate thereof, wherein the composition is a solution, wherein the solution has a pH of from about 6.5 to about 8.
2. The composition according to the above 1, further comprising a polyoxyethylene sorbitan fatty acid esters comprising polysorbate 80.
3. The composition according to the above 2, wherein the polyoxyethylene sorbitan fatty acid ester is polysorbate 80.
4. The composition according to the above 1, further comprising a non-ionic solubilizer such as polyoxyl-15-hydroxystearate.
5. A stable pharmaceutical composition comprising bromfenac or a pharmacologically acceptable salt or hydrate thereof, wherein the composition is a solution, is stable, and comprises at least one of:
   a. an polyoxyethylene sorbitan fatty acid esters such as polysorbate 80 or;
   b. a non-ionic solubilizer such as polyoxyl-15-hydroxystearate and wherein the composition does not comprise any of the following;
   c. an alkyl aryl polyether alcohol type polymer;
   d. a polyethylene glycol fatty acid ester or;
   e. an antioxidant.
6. The bromfenac composition according to the above 1 to 5 that does not comprise an alkyl aryl polyether alcohol type polymer, a polyethylene glycol fatty acid ester, and an antioxidant.
7. The bromfenac composition according to the above 1 to 5 that does not comprise tyloxapol or polyethylene glycol monostearate such as polyoxyl 40 stearate.
8. The bromfenac composition of claim 7, that does not comprise an alkyl aryl polyether alcohol type polymer, a polyethylene glycol fatty acid ester, and an antioxidant.
9. The bromfenac composition according to the above 1 to 5 that does not comprise a sulfite anti-oxidant.
10. The bromfenac solution according to the above 1 to 5 does not comprise a borate buffer.
11. The bromfenac solution according to the above 1 to 5, wherein the solution is aqueous and comprises citrate buffer.
12. The bromfenac solution according to the above 1 to 5 that has a pH from about 6.5 to about 8.
13. The bromfenac solution according to the above 12 that has a pH from about 7.2 to about 7.8.
14. A bromfenac solution according to the above 1 to 5, wherein the solution is contained in a unit dose kit form.
15. A bromfenac solution according to the above 1 to 5, wherein the solution is contained in a multi dose kit form.
16. An aqueous solution according to the above 1 to 5, which is an aqueous solution comprising bromfenac sodium salt or a hydrate thereof, wherein the concentration of the bromfenac sodium salt or the hydrate thereof is from about 0.01 to about 0.1 w/v %.
17. The bromfenac composition according to the above 1 to 5 wherein benzalkonium chloride is contained as a preservative.
18. The bromfenac composition according to the above 1, 5 and 16 wherein the hydrate is at least one selected from a 1/2 hydrate, 1 hydrate and 3/2 hydrate.
19. A bromfenac composition according to the above 1 to 5 wherein the composition is a solution and is applied once a day to each eye in need thereof
20. A bromfenac composition according to the above 1 to 5 wherein the composition is a solution and is applied twice a day to each eye in need thereof
21. A method for stabilizing an aqueous solution of bromfenac or its pharmacologically acceptable salt or a hydrate thereof, comprising combining bromfenac or a pharmacologically acceptable salt or hydrate thereof, water, and a solubilizer, wherein the solubilizer is selected from a group consist of either a polyoxyethylene sorbitan fatty acid esters such as polysorbate 80 or a non-ionic soulbilizer such as polyoxyl-15-hydroxystearate according to any of the above 1 to 19.
22. A process of preparing a stable, aqueous solution according to the above 1 to 5 and 20.
23. The aqueous solution prepared by the method according to the above 22.
24. The aqueous solution according to the above 22, which exhibits stability when stored at stress conditions at 50° C. for 4 weeks, when stored at accelerated conditions at 40° C. for 6 months, or when stored at long term conditions.
25. The aqueous solution according to the above 24, wherein said stability includes one or more of the following:
   a) at least 90% remaining bromfenac;
   b) not more than 3.0% total impurity;
   c) not more than 0.8% impurity A;
   d) not more than 0.8% impurity B.
26. A method of treating ocular inflammation or pain in a patient in need thereof, comprising administering to an eye of the patient an effective amount of an aqueous solution according to any of the above 25.
27. The bromfenac solution according to the above 25, which upon storage for 6 months at accelerated conditions at 40° C. at no more than 25% relative humidity, comprises less than 0.8% of impurity B {(7-(4-bromobenzoyl) indoline-2,3-dione} of active ingredient.
28. A method of treating ocular inflammation or pain in a patient in need thereof, comprising administering to an eye of the patient an effective amount of a stable pharmaceutical aqueous solution comprising bromfenac, or a pharmaceutically acceptable salt or hydrate thereof; further comprising a polyoxyethylene sorbitan fatty acid ester and/or a polyoxyl-15-hydroxystearate; wherein the solution has a pH of from about 6.5 to about 8; and wherein the solution does not comprise any of an alkyl aryl polyether alcohol type polymer, a polyethylene glycol fatty acid ester, and an antioxidant.
29. Use of a composition for treating ocular inflammation or pain in a patient in need thereof, comprising administering to an eye of the patient an effective amount of an aqueous solution according to any of the above 25.
30. Use of a composition for treating ocular inflammation or pain in a patient in need thereof, comprising administering to an eye of the patient an effective amount of a stable pharmaceutical aqueous solution comprising bromfenac, or a pharmaceutically acceptable salt or hydrate thereof; further comprising a polyoxyethylene sorbitan fatty acid ester and/or a polyoxyl-15-hydroxystearate; wherein the solution has a pH of from about 6.5 to about 8; and wherein the solution does not comprise any of an alkyl aryl polyether alcohol type polymer, a polyethylene glycol fatty acid ester, and an antioxidant.
   The present invention provides a method of using the inventive compositions for treating ocular inflammation and pain after cataract surgery.

In a preferred embodiment, the present invention provides a process of preparing a stable, aqueous solution wherein the composition is prepared by a process comprising:

1. Add quantity of purified water approximately 80% of the batch size in a container, e.g., a stainless steel vessel.

2. Add one component of a buffer system (e.g., Sodium citrate dihyrate) to step 1 under stirring and mix until dissolved.

3. Add mannitol to step 2 under stirring and mix until dissolved.

4. Add sodium chloride to step 3 under stirring and mix until dissolved.

5. Add chelating agent (e.g., disodium edetate) to step 4 under stirring and mix until dissolved.

6. Add surfactant (e.g., a polyoxyethylene sorbitan fatty acid esters such as polysorbate 80 or a non-ionic solubilizer such as polyoxyl-15-hydroxystearate) to step 5 under stirring and mix until dissolved.

7. Add preservative (e.g., BKC) to step 6 under stirring and mix until dissolved.

8. Add NSAID (e.g., bromfenac sodium) to step 7 under stirring and mix until dissolved.

9. Check pH of the solution; adjust if necessary to pH 7.8.

10. Make up the volume to 100% of the batch size with purified water and stir for 5 minutes.

11. Finally the solution is filtered through 0.22 micron filter.

As can be determined by one of skill in the art using the present disclosure as a guide, the order of the above steps can be changed, and two or more steps may be combined.

### Stability Studies:

The stable, aqueous formulations (batch numbers 35B & 35C) of Bromfenac are prepared with ranges of ingredients as shown in Table 1, and are exposed to stress conditions at 50° C. for initial (0 days), 2 to 4 weeks; at accelerated conditions at 40° C. for 1, 2, 3 & 6 months and finally exposed to long term conditions at 25° C. for 3 months and 6 months to determine the stability of the proposed formulations of the present invention.

One more formulation (Formulation 2) of the present invention is exposed to stress conditions at 50° C. for initial (0 days), 2 weeks and 4 weeks for stress stability at 50° C. in the dark.

A comparative study is initiated to determine the stability of present invention formulations with a marketed product PROLENSA™ herein defined as “Control”.

A non-accelerated or accelerated study method may be used to test stability. A preferred stress (or accelerated) study comprises placing the composition/solution is filled in Opaque LDPE vial with LDPE nozzle and HDPE cap, packing in secondary packaging material, and maintaining at 50° C. in the dark. Impurities are measured by HPLC for initial (0 days), 2 weeks and 4 weeks.

As understood by those of skill in the art, when the NSAID comprises bromfenac, the impurities preferably measured include “Impurity A” as (7-(4-Bromobenzoyl)-1,3-dihydro-2H-indol-2-one), “Impurity B” as (7-(4-bromobenzoyl) indoline-2,3-dione) and the total impurities, as well as identification of the amount of the highest unknown impurity.

TABLE 1

| | Ranges varied in % w/v | Ranges varied |
|---|---|---|
| | (batch numbers 35B & | in % w/v |
| Ingredients | 35C) | (Formulation 2) |
| Bromfenac Sodium | 0.0805 | 0.0805 |
| Edetate Disodium | 0.02-0.15 | 0.02-0.15 |
| Sodium citrate dihydrate | 0.05-0.4 | 0.05-0.4 |
| Mannitol | 0.2-4.0 | 0.2-4.0 |
| Sodium chloride | 0.05-0.6 | 0.05-0.6 |
| Benzalkonium chloride | 0.0005-0.5 | 0.0005-0.5 |
| Polysorbate 80 | 0.005-0.2 | — |
| Polyoxyl-15-Hydroxystearate | — | 0.005-0.2 |
| Sodium hydroxide | QS | QS |
| MilliQ water | QS | QS |
| pH | 7.8 | 7.8 |

### Results and Observations:

The control formulation (PROLENSA™) is evaluated for bromfenac content and related substances at initial (0 days), 2 weeks and 4 weeks for stress stability at 50° C.; at accelerated conditions at 40° C. for 1, 2, 3 & 6 months in the dark. Results are shown in Table 2.

The formulations (batch numbers 35B & 35C) of the present invention are evaluated for bromfenac content and related substances at initial (0 days), 2 weeks and 4 weeks for stress stability at 50° C.; at accelerated conditions at 40° C. for 1, 2, 3 & 6 months and finally exposed to long term conditions at 25° C. for 3 months and 6 months in the dark. Results are shown in Table 3 for batch numbers 35B and Table 4 for batch numbers 35C.

One more formulation (Formulation 2) of the present invention is evaluated for bromfenac content and related substances at initial (0 days), 2 weeks and 4 weeks for stress stability at 50° C. in the dark. Results are shown in Table 5 for formulation 2.

TABLE 2
{Control formulation (PROLENSA ™)}

| | Proposed | | Stress conditions | | Accelerated conditions | | | |
|---|---|---|---|---|---|---|---|---|
| | Shelf life | | 2 | 4 | 1 | 2 | 3 | 6 |
| | Specs. | | Weeks/ | Weeks/ | Month/ | Month/ | Month/ | Month/ |
| Parameters | Limits | Initial | 50° C. | 50° C. | 40° C. | 40° C. | 40° C. | 40° C. |
| Appearance | CGYS | CGYS | CGYS | CGYS | CGYS | CGYS | CGYS | CGYS |
| pH | 7.2-8.0 | 7.78 | NA | 7.83 | NA | NA | 7.80 | 7.78 |
| Osmolality | 280-340 | 310 | NA | 328 | NA | NA | 320 | 335 |
| (mOsmol/kg) | | | | | | | | |

Related Substances

| | | | | | | | | |
|---|---|---|---|---|---|---|---|---|
| Impurity A * | NMT 0.8 | 0.02 | 0.01 | ND | ND | ND | ND | BQL |
| Impurity B ^{$} | NMT 0.8 | BDL | 0.14 | 0.16 | 0.07 | 0.25 | 0.49 | 1.47 |
| Highest | NMT 0.8 | ND | ND | 0.12 | 0.06 | 0.08 | 0.12 | 0.42 |
| unknown | | | | | | | | |
| impurity | | | | | | | | |
| Total | NMT 3.0 | 0.02 | 0.15 | 0.33 | 0.13 | 0.40 | 0.77 | 2.89 |
| impurities | | | | | | | | |
| Assay of | 90.0-110.0 | 99.1 | NA | 99.3 | NA | NA | 97.0 | 92.7 |
| bromfenac | | | | | | | | |
| free acid | | | | | | | | |
| Content of | 75-120 | NA | NA | NA | NA | NA | NA | NA |
| BKC | | | | | | | | |
| Content of | 70-115 | NA | NA | NA | NA | NA | NA | NA |
| EDTA | | | | | | | | |

* 7-(4-Bromobenzoyl)-1,3-dihydro-2H-indol-2-one
^{$} 7-(4-bromobenzoyl) indoline-2,3-dione
ND: Not Detected
NA: Not Analyzed
BDL: Below Disregard Limit
BQL: Below Quantification Limit

TABLE 3
(Batch no. PR3F048-35B)

| | | | | | | | | | Long term | |
|---|---|---|---|---|---|---|---|---|---|---|
| | Proposed | | Stress conditions | | Accelerated conditions | | | | conditions | |
| | Shelf life | | 2 | 4 | 1 | 2 | 3 | 6 | 3 | 6 |
| | Specs. | | Weeks/ | Weeks/ | Month/ | Month/ | Month/ | Month/ | Month/ | Month/ |
| Parameters | Limits | Initial | 50° C. | 50° C. | 40° C. | 40° C. | 40° C. | 40° C. | 25° C. | 25° C. |
| Appearance | CGYS | CGYS | CGYS | CGYS | CGYS | CGYS | CGYS | CGYS | CGYS | CGYS |
| pH | 7.2-8.0 | 7.59 | 7.52 | 7.59 | 7.58 | 7.57 | 7.47 | 7.47 | 7.35 | 7.39 |
| Osmolality | 280-340 | 304 | 309 | 321 | 310 | 313 | 320 | 324 | 310 | 312 |
| (mOsmol/kg) | | | | | | | | | | |

Related Substances

| | | | | | | | | | | |
|---|---|---|---|---|---|---|---|---|---|---|
| Impurity A * | NMT 0.8 | 0.01 | 0.04 | 0.06 | 0.06 | 0.09 | 0.11 | 0.12 | 0.13 | 0.17 |
| Impurity B ^{$} | NMT 0.8 | 0.08 | 0.08 | 0.08 | 0.09 | 0.08 | 0.08 | 0.50 | 0.08 | 0.07 |
| Highest | NMT 0.8 | ND | ND | 0.07 | ND | BDL | 0.09 | 0.12 | BDL | 0.08 |
| unknown | | | | | | | | | | |
| impurity | | | | | | | | | | |
| Total | NMT 3.0 | 0.09 | 0.12 | 0.19 | 0.15 | 0.17 | 0.28 | 0.74 | 0.21 | 0.32 |
| impurities | | | | | | | | | | |
| Assay of | 90.0-110.0 | 98.8 | 102.3 | 103.1 | 99. | 100.4 | 100.7 | 104.1 | 99.2 | 101.0 |
| bromfenac | | | | | | | | | | |
| free acid | | | | | | | | | | |
| Content of | 75-120 | 96.6 | 104.0 | 107.0 | 100.0 | 104.4 | 103.6 | 104.0 | 100.4 | 101.2 |
| BKC | | | | | | | | | | |
| Content of | 70-115 | 100.6 | 102.0 | 104.1 | 101.6 | 104.0 | 104.4 | 106.9 | 102.0 | 102.2 |
| EDTA | | | | | | | | | | |

* 7-(4-Bromobenzoyl)-1,3-dihydro-2H-indol-2-one
^{$} 7-(4-bromobenzoyl) indoline-2,3-dione
ND: Not Detected
BDL: Below Disregard Limit
NA: Not Analyzed
CGYS: Clear, greenish yellow solution

TABLE 4
(Batch no. PR3F048-35C)

| | | | | | | | | | Long term | |
|---|---|---|---|---|---|---|---|---|---|---|
| | Proposed | | Stress conditions | | Accelerated conditions | | | | conditions | |
| | Shelf life | | 2 | 4 | 1 | 2 | 3 | 6 | 3 | 6 |
| | Specs. | | Week/ | Week/ | Month/ | Month/ | Month/ | Month/ | Month/ | Month/ |
| Parameters | Limits | Initial | 50° C. | 50° C. | 40° C. | 40° C. | 40° C. | 40° C. | 25° C. | 25° C. |
| Appearance | CGYS | CGYS | CGYS | CGYS | CGYS | CGYS | CGYS | CGYS | CGYS | CGYS |
| pH | 7.2-8.0 | 7.88 | 7.63 | 7.59 | 7.58 | 7.56 | 7.48 | 7.49 | 7.47 | 7.47 |
| Osmolality | 280-340 | 304 | 307 | 317 | 310 | 313 | 317 | 331 | 310 | 317 |
| (mOsmol/kg) | | | | | | | | | | |

Related Substances

| | | | | | | | | | | |
|---|---|---|---|---|---|---|---|---|---|---|
| ImpurityA * | NMT 0.8 | 0.03 | 0.04 | 0.04 | 0.05 | 0.07 | 0.09 | 0.11 | 0.12 | 0.13 |
| Impurity B ^{$} | NMT 0.8 | 0.08 | 0.08 | 0.08 | 0.09 | 0.08 | 0.07 | 0.43 | 0.08 | 0.06 |
| Highest | NMT 0.8 | ND | ND | 0.05 | ND | BDL | 0.09 | 0.11 | BDL | 0.08 |
| unknown | | | | | | | | | | |
| impurity | | | | | | | | | | |
| Total | NMT 3.0 | 0.11 | 0.12 | 0.17 | 0.14 | 0.15 | 0.25 | 0.65 | 0.20 | 0.27 |
| impurities | | | | | | | | | | |
| Assay of | 90.0-110.0 | 98.8 | 101.4 | 101.8 | 98.5 | 100.1 | 101.4 | 103.4 | 99.2 | 101.0 |
| bromfenac | | | | | | | | | | |
| free acid | | | | | | | | | | |
| Content of | 7-120 | 102.2 | 102.8 | 103.2 | 101.9 | 103.4 | 103.4 | 103.4 | 100.8 | 101.4 |
| BKC | | | | | | | | | | |
| Content of | 70-115 | 101.0 | 100.1 | 102.7 | 100.4 | 102.4 | 104.1 | 107.2 | 102.3 | 100.9 |
| EDTA | | | | | | | | | | |

* 7-(4-Bromobenzoyl)-1,3-dihydro-2H-indol-2-one
^{$} 7-(4-bromobenzoyl) indoline-2,3-dione
ND: Not Detected
BDL: Below Disregard Limit
CGYS: Clear, greenish yellow solution

TABLE 5
(Formulation 2)

| | Initial | | |
|---|---|---|---|
| | sample | 2 weeks, | 4 weeks, |
| Parameters (Formulation 2) | results | 50° C. | 50° C. |
| Description | CGYS | CGYS | CGYS |
| pH | 7.43 | 7.43 | 7.35 |
| Osmolality (mOsmol/kg) | 305 | 307 | 314 |

Related Substance

| | | | |
|---|---|---|---|
| Impurity A | 0.02% | 0.07% | 0.10% |
| Highest unknown impurity | 0.08% | 0.10% | 0.22% |
| Total impurities | 0.17% | 0.17% | 0.32% |
| Specified unidentified | Not detected | Not detected | Not detected |
| impurity at RRT ~0.30 | | | |
| Assay (bromfenac free acid) | 101.50% | 102.00% | 100.70% |

### Results of Control Formulation (Prolensa™) (Batch—178392):

### The Results of Stress Stability at 50° C. for 4 Weeks for “Control” Formulation:

The Bromfenac content is measured and found to be 99.3% (Limit: 90.0-110.0%) which is in the acceptable limit range, the highest unknown impurity is measured and found to be 0.12% (Limit: NMT 0.8%) which is in the acceptable limit range and total impurity is measured and found to be 0.33% (Limit: NMT 3.0%) which is in the acceptable limit range.

### The Results of Accelerated Stability at 40° C. for 6 Months for “Control” Formulation:

The Bromfenac content is measured and found to be 92.7% (Limit: 90.0-110.0%) which is in the acceptable limit range, the highest unknown impurity is measured and found to be 0.42% (Limit: NMT 0.8%) which is in the acceptable limit range and total impurity is measured and found to be 2.89% (Limit: NMT 3.0%) which is in the acceptable limit range.

### Results of Present Invention Formulation (Batch—35B):

### The Results of Stress Stability at 50° C. for 4 Weeks for Present Invention Formulation (Batch—35B):

The Bromfenac content is measured and found to be 103.1% (Limit: 90.0-110.0%) which is in the acceptable limit range, the highest unknown impurity is measured and found to be 0.07% (Limit: NMT 0.8%) which is in the acceptable limit range and total impurity is measured and found to be 0.19% (Limit: NMT 3.0%) which is in the acceptable limit range.

### The Results of Accelerated Stability at 40° C. for 6 Months for Present Invention Formulation (Batch—35B):

The Bromfenac content is measured and found to be 104.1% (Limit: 90.0-110.0%) which is in the acceptable limit range, the highest unknown impurity is measured and found to be 0.12% (Limit: NMT 0.8%) which is in the acceptable limit range and total impurity is measured and found to be 0.74% (Limit: NMT 3.0%) which is in the acceptable limit range.

### The Results of Long Term Stability at 25° C. for 6 Months for Present Invention Formulation (Batch—35B):

The Bromfenac content is measured and found to be 101.0% (Limit: 90.0-110.0%) which is in the acceptable limit range, the highest unknown impurity is measured and found to be 0.08% (Limit: NMT 0.8%) which is in the acceptable limit range and total impurity is measured and found to be 0.32% (Limit: NMT 3.0%) which is in the acceptable limit range.

### Results of Present Invention Formulation (Batch—35C):

### The Results of Stress Stability at 50° C. for 4 Weeks for Present Invention Formulation (Batch—35C):

The Bromfenac content is measured and found to be 101.8% (Limit: 90.0-110.0%) which is in the acceptable limit range, the highest unknown impurity is measured and found to be 0.05% (Limit: NMT 0.8%) which is in the acceptable limit range and total impurity is measured and found to be 0.17% (Limit: NMT 3.0%) which is in the acceptable limit range.

### The Results of Accelerated Stability at 40° C. for 6 Months for Present Invention Formulation (Batch—35C):

The Bromfenac content is measured and found to be 103.4% (Limit: 90.0-110.0%) which is in the acceptable limit range, the highest unknown impurity is measured and found to be 0.11% (Limit: NMT 0.8%) which is in the acceptable limit range and total impurity is measured and found to be 0.65% (Limit: NMT 3.0%) which is in the acceptable limit range.

### The Results of Long Term Stability at 25° C. for 6 Months for Present Invention Formulation (Batch—35C):

The Bromfenac content is measured and found to be 101.0% (Limit: 90.0-110.0%) which is in the acceptable limit range, the highest unknown impurity is measured and found to be 0.08% (Limit: NMT 0.8%) which is in the acceptable limit range and total impurity is measured and found to be 0.27% (Limit: NMT 3.0%) which is in the acceptable limit range.

### Results of Present Invention Formulation (Formulation 2):

### The Results of Stress Stability at 50° C. for 4 Weeks for Polyoxyl 15 Hydroxy Stearate (Formulation 2):

The Bromfenac content is measured to be 100.7% (Limit: 90.0-110.0%) which is in the acceptable limit range, the highest unknown impurity is measured to be 0.22% (Limit: NMT 0.8%) which is in the acceptable limit range and total impurity is measured to be 0.32% (Limit: NMT 3.0%) which is in the acceptable limit range.

All the following formulations (batch numbers 35B; 35C & formulation 2) were found to be stable at all stability conditions.

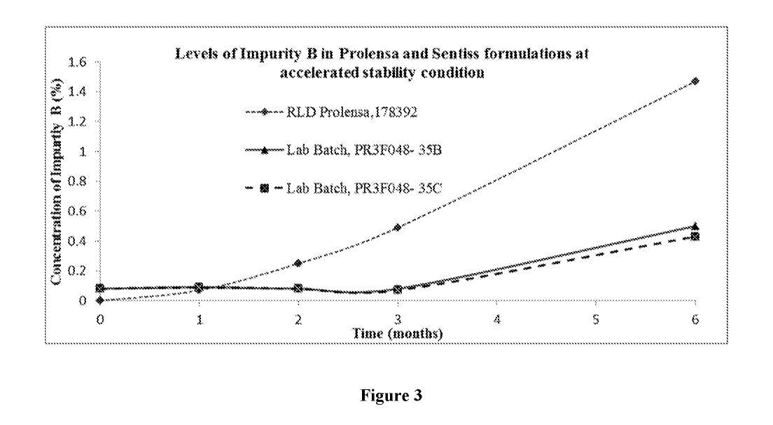

Based on the stability studies conducted on control formulation (PROLENSA™) (BATCH—178392) and formulations (batch numbers 35B; 35C) of the present invention, it was observed that level of Impurity B in present invention formulations is approximately ⅓rd of the level in Control formulation at 6 month accelerated condition (Levels of Impurity B level as shown in FIGS. 3—0.43% (batch 35C) & 0.50% (batch 35B) of present invention formulation verses 1.47% of Control formulation—PROLENSA™).

Subsequently, the levels of total impurities are also less in present invention formulations in comparison to Control formulation at 6M accelerated condition (0.65% (batch 35C) in Sentiss formulation verses 2.89% in Control formulation). All other parameters were well within the specifications.

### Animal Efficacy Studies:

The objective of the study was to evaluate and compare ocular anti-inflammatory activity of different formulations in Wistar rats following ocular installation. The summary of Animal efficacy studies is in table 6.

TABLE 6
Summary of Animal Studies

| | |
|---|---|
| Clinical signs | All animals were normal, except diarrhoea in few animals due to |
| | endotoxin and recovered during next observation. |
| Mortality | All animals survived throughout the experiment |

Ophthalmoscopic examinations

| | |
|---|---|
| Vasodilatation | Dilatation of blood vessels was assessed on the basis of the |
| (% of the mean of negative | hyperaemia developed. The ophthalmoscopic observation data |
| control) | have been reported in table 8 and same has been plotted in FIG. |
| | 2. |
| | Group I - Negative control (Normal saline - 10 μL/eye) |
| | Group II (036A) - Present Invention formulation (10 μL/eye) |
| | Group III (178392) - Innovator's product (PROLENSA ™) (10 μL/ |
| | eye) |
| | Groups II vs Group III: |
| | Comparable (p <1.000 @ 24 hrs & 0.794 @ 48 hrs) |
| Presence of flare & corneal | Scored zero (0) as no flare observed and no corneal damage in |
| observations | any of the dose groups |
| Others | No abnormalities detected in any of the dose groups |

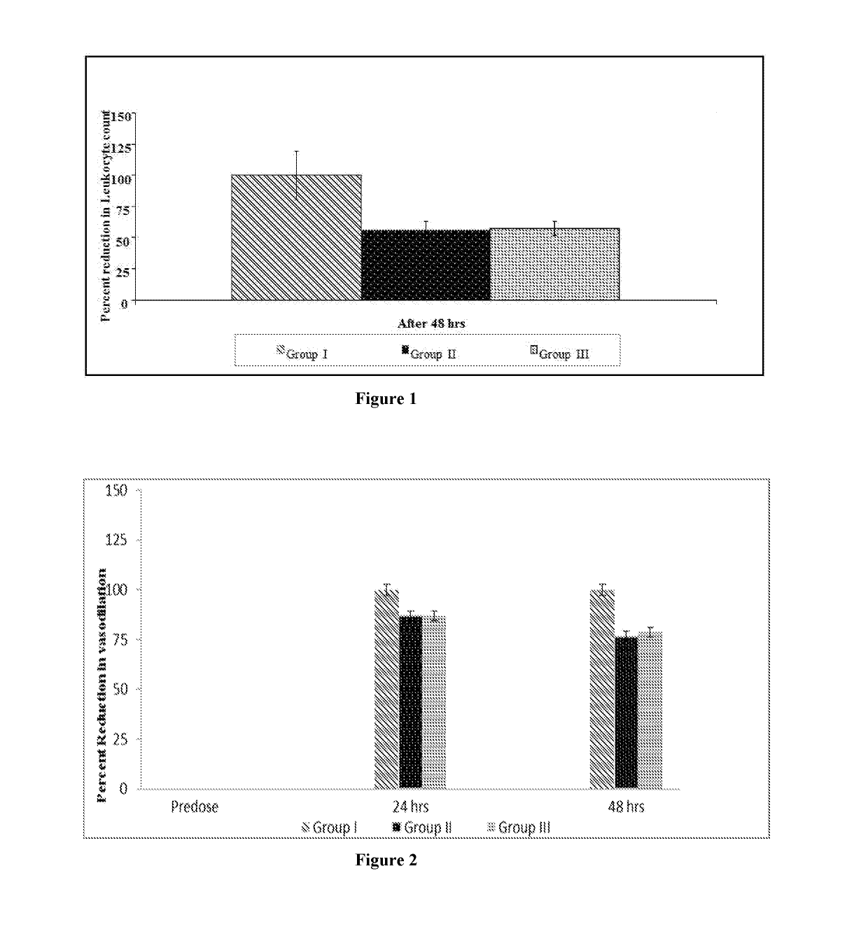

Leukocyte counts (from aqueous humor)

| | |
|---|---|
| Counts | Leucocyte count: The leukocyte count data have been |
| (% of the mean of negative | reported in table 7 and same has been plotted in FIG. 1. |
| control) | Group I: 86.40 ± 16.71 (100%) |
| | Group II: 48.20 ± 6.29 (55.79%) |
| | Group III: 49.35 ± 4.86 (57.12%) |
| | Groups II (55.79%) vs Group I: |
| | Significant reduction in Group II (p <0.039 @ 48 hrs) |
| | Groups III (57.12%) vs Group I: |
| | Significant reduction in Group III (p <0.040 @ 48 hrs) |
| | Groups II vs Group III: Comparable (p <0.886 @ 48 hrs) |

TABLE 7
Comparative leukocyte counts between Group I (Negative control),
Group II (Present Invention formulation - 036A) & Group III (Innovator
product - PROLENSA ™).

| Groups | Parameters | Time point: 48 hrs |
|---|---|---|
| Group I | % leukocytes (leukocytes count) | 100% (86.4/mm²) |
| Group II | % leukocytes (leukocytes count) | 55.79% (48.20/mm²) |
| Group III | % leukocytes (leukocytes count) | 57.12% (49.35/mm²) |

TABLE 8
Comparative vasodilation reduction between Group I (Negative
control), Group II (Present Invention formulation - 036A) & Group III
(Innovator product - PROLENSA ™).

| | Parameters | Time points | | |
|---|---|---|---|---|
| Groups | Average | Pre-dose | 24 hrs | 48 hrs |
| Group I | % inflam- | 0.00% (0.00) | 100.00% (2.70) | 100.00% (2.80) |
| | mation | | | |
| | (Dilatation | | | |
| | score) | | | |
| Group II | % inflam- | 0.00% (0.00) | 87.04% (2.35) | 76.79% (2.15) |
| | mation | | | |
| | (Dilatation | | | |
| | score) | | | |
| Group | % inflam- | 0.00% (0.00) | 87.04% (2.35) | 78.57% (2.20) |
| III | mation | | | |
| | (Dilatation | | | |
| | score) | | | |

CONCLUSION: Based on the observations obtained from this study, it is concluded that both the formulations {batch numbered Group II (Present Invention formulation) 036A & & Group III (Innovator product—PROLENSA™-178392} have shown statistically significant efficacy on ocular inflammation when administered to Wistar rats by ocular route and this effect of Group II (036A) was statistically comparable to the efficacy of Group III (178392).

### Examples

The scope of the present invention is illustrated by the following examples which are not meant to restrict the scope of the invention in any manner whatsoever.

The term ‘q.s.’ wherever appears in the examples is an abbreviation for ‘quantity sufficient’ which is the amount of the excipient in such quantities that is just sufficient for its use in the composition of the present invention.

The term ‘° C.’ wherever appears is an abbreviation for “Degree Celsius” and the term “NMT” wherever appears is an abbreviation for “Not More Than”.

Formulas I exhibit good stability following the stress stability test at 50° C. for initial (0 days), 2 weeks and 4 weeks; at accelerated conditions at 40° C. for 1, 2, 3 & 6 months and finally exposed to long term conditions at 25° C. for 3 months and 6 months.

Formulas II exhibit good stability following the stress stability test at 50° C. for initial (0 days), 2 weeks and 4 weeks.

### Formula I:

| Ingredients | % w/v |
|---|---|
| Bromfenac Sodium | 0.0805 |
| Edetate disodium | 0.1 |
| Polysorbate 80 | 0.01 |
| Sodium citrate dihyrate | 0.292 |
| Mannitol | 2.0 |
| Sodium chloride | 0.45 |
| BKC | 0.005 |
| Sodium hydroxide | q.s. |
| MilliQ water | q.s. |
| pH | 7.8 |

### Method:

1. Add quantity of purified water approximately 80% of the batch size in a container, e.g., a stainless steel vessel.

2. Add one component of a buffer system (e.g., Sodium citrate dihyrate) to step 1 under stirring and mix until dissolved.

3. Add mannitol to step 2 under stirring and mix until dissolved.

4. Add sodium chloride to step 3 under stirring and mix until dissolved.

5. Add chelating agent (e.g., disodium edetate) to step 4 under stirring and mix until dissolved.

6. Add surfactant (e.g., Polysorbate 80) to step 5 under stirring and mix until dissolved.

7. Add preservative (e.g., BKC) to step 6 under stirring and mix until dissolved.

8. Add NSAID (e.g., bromfenac sodium) to step 7 under stirring and mix until dissolved.

9. Check pH of the solution; adjust if necessary to pH 7.8.

10. Make up the volume to 100% of the batch size with purified water and stir for 5 minutes.

11. Finally the solution is filtered through 0.22 micron filter.

### Formula II:

| Ingredients | % w/v |
|---|---|
| Bromfenac Sodium | 0.0805 |
| Edetate disodium | 0.1 |
| Kolliphore HS 15 (Polyoxyl 15 | 0.01 |
| hydroxy stearate) | |
| Sodium citrate dihyrate | 0.292 |
| Mannitol | 2.0 |
| Sodium chloride | 0.45 |
| BKC | 0.005 |
| Sodium hydroxide | q.s. |
| MilliQ water | q.s. |
| pH | 7.8 |

### Method:

1. Add quantity of purified water approximately 80% of the batch size in a container, e.g., a stainless steel vessel.

2. Add one component of a buffer system (e.g., Sodium citrate dihyrate) to step 1 under stirring and mix until dissolved.

3. Add mannitol to step 2 under stirring and mix until dissolved.

4. Add sodium chloride to step 3 under stirring and mix until dissolved.

5. Add chelating agent (e.g., disodium edetate) to step 4 under stirring and mix until dissolved.

6. Add surfactant (e.g., Kolliphore HS 15 (Polyoxyl 15 hydroxy stearate)) to step 5 under stirring and mix until dissolved.

7. Add preservative (e.g., BKC) to step 6 under stirring and mix until dissolved.

8. Add NSAID (e.g., bromfenac sodium) to step 7 under stirring and mix until dissolved.

9. Check pH of the solution; adjust if necessary to pH 7.8.

10. Make up the volume to 100% of the batch size with purified water and stir for 5 minutes.

11. Finally the solution is filtered through 0.22 micron filter.

### Comparative Example

Polysorbate 80 concentrations at or above 0.15% w/v may be unstable during stress_stability at 50° C. for four weeks as shown in table 5.

| Ingredients | % w/v |
|---|---|
| Bromfenac Sodium | 0.0805 |
| Edetate disodium | 0.1 |
| Polysorbate 80 | 0.15 |
| Sodium citrate dihyrate | 0.292 |
| Mannitol | 2.0 |
| Sodium chloride | 0.45 |
| Sodium hydroxide | q.s. |
| BKC | 0.005 |
| Purified water (MilliQ water) | q.s. |
| pH | 7.8 |

TABLE 5

| Parameters (Polysorbate 80) | 4 weeks, 50° C. |
|---|---|
| Description | Yellow colored solution |
| pH | 7.14 |
| Osmolality (mOsmol/kg) | 337 |

Related Substance

| | |
|---|---|
| Impurity A | 0.23% |
| Highest unknown impurity | 2.54% |
| Total impurities | 6.64% |
| Assay (bromfenac free acid) | 75.80% |

After four weeks of the stress test, Bromfenac content is measured to be 75.80% (Limit: 90.0-110.0%), which is below the acceptable limit range, the highest unknown impurity is measured to be 2.54% (Limit: NMT 1.0%), which is above the acceptable limit range and total impurity is measured to be 6.64% (Limit: NMT 3.0%), which is above the acceptable limit range.

The comparative Stability studies between control and present inventions show that the ingredients used in the formulations have varied effects on the stability of the formulations investigated when measured at different time periods (at initial (0 days), 2 weeks and 4 weeks) at 50° C.; at accelerated conditions at 40° C. for 1, 2, 3 & 6 months and finally exposed to long term conditions at 25° C. for 3 months and 6 months.

The inventors of the present invention with expenditure of intellectual effort and careful experimentation have identified unique concentration of solubilizers such as Polysorbate 80 that resolves the hazinesss of the formulations of the present invention but does not affect degradation of bromfenac, in spite of not using antioxidants.

Formulations of the present invention are unexpectedly as stable as that of control at a pH 7.8, without the need for, or use of, antioxidants such as sulfite(s) such as sodium sulfite, potassium sulfite, and the like, and/or with the addition of pharmaceutically acceptable solubilizers such as either an polyoxyethylene sorbitan fatty acid esters such as polysorbate 80 or a non-ionic solubilizer such as polyoxyl-15-hydroxystearate.

Even based on the stability studies conducted on control formulation (PROLENSA™) (BATCH—178392) and formulations (batch numbers 35B; 35C) of the present invention, it was observed that level of Impurity B in present invention formulations is approximately ⅓rd of the level in Control formulation at 6 month accelerated condition (Levels of Impurity B level as shown in FIGS. 3—0.43% (batch 35C) & 0.50% (batch 35B) of present invention formulation verses 1.47% of Control formulation—PROLENSA™). This demonstrates that the present invention formulations are unexpectedly as stable as and even more stable than that of control formulation (PROLENSA™) as shown in FIG. III.

The claim:

1. A stable pharmaceutical composition comprising bromfenac, or a pharmaceutically acceptable salt or hydrate thereof, wherein the composition is a solution; and wherein the solution has a pH of from about 6.5 to about 8.

2. The composition of claim 1, further comprising a polyoxyethylene sorbitan fatty acid ester comprising polysorbate 80.

3. The composition of claim 2, wherein the polyoxyethylene sorbitan fatty acid ester is polysorbate 80.

4. The composition of claim 1, further comprising a non-ionic solubilizer such as polyoxyl-15-hydroxystearate.

5. A stable pharmaceutical composition comprising bromfenac or a pharmacologically acceptable salt or hydrate thereof, wherein the composition is a solution, is stable, and comprises at least one of:
a. an polyoxyethylene sorbitan fatty acid esters such as polysorbate 80 or;
b. a non-ionic solubilizer such as polyoxyl-15-hydroxystearate; and wherein the composition does not comprise any of the following;
c. an alkyl aryl polyether alcohol type polymer;
d. a polyethylene glycol fatty acid ester or;
e. an antioxidant.

6. The bromfenac composition of any of claims 1 to 5 that does not comprise an alkyl aryl polyether alcohol type polymer, a polyethylene glycol fatty acid ester, and an antioxidant.

7. The bromfenac composition of any of claims 1 to 5 that does not comprise tyloxapol or polyethylene glycol monostearate such as polyoxyl 40 stearate.

8. The bromfenac composition of claim 7, that does not comprise an alkyl aryl polyether alcohol type polymer, a polyethylene glycol fatty acid ester, and an antioxidant.

9. The bromfenac composition of any of claims 1 to 5, that does not comprise a sulfite anti-oxidant.

10. The bromfenac solution of any of claims 1 to 5, that does not comprise a borate buffer.

11. The bromfenac solution of any of claims 1 to 5, wherein the solution is aqueous and comprises citrate buffer.

12. The bromfenac solution of any of claims 1 to 5 that has a pH from about 6.5 to about 8.

13. The bromfenac solution of claim 12 that has a pH from about 7.2 to about 7.8.

14. The bromfenac solution of any of claims 1 to 5, wherein the solution is contained in a unit dose kit form.

15. The bromfenac solution of any of claims 1 to 5, wherein the solution is contained in a multi dose kit form.

16. The bromfenac solution of any of claims 1 to 5, which is an aqueous solution comprising bromfenac sodium salt or a hydrate thereof, wherein the concentration of the bromfenac sodium salt or the hydrate thereof is from about 0.01 to about 0.1 w/v %.

17. The bromfenac composition of any of the claims 1 to 5 wherein benzalkonium chloride is contained as a preservative.

18. The bromfenac composition of any of claims 1, 5 and 16 wherein the hydrate is at least one selected from a 1/2 hydrate, 1 hydrate and 3/2 hydrate.

19. The bromfenac composition of any of claims 1 to 5 wherein the composition is a solution and is applied once a day to each eye in need thereof.

20. The bromfenac composition of any of the claims 1 to 5 wherein the composition is a solution and is applied twice a day to each eye in need thereof.

21. A method for stabilizing an aqueous solution of bromfenac or its pharmacologically acceptable salt or a hydrate thereof, comprising combining bromfenac or a pharmacologically acceptable salt or hydrate thereof, water, and a solubilizer, wherein the solubilizer is selected from a group consist of either a polyoxyethylene sorbitan fatty acid esters such as polysorbate 80 or a non-ionic soulbilizer such as polyoxyl-15-hydroxystearate.

22. A process of preparing a stable, aqueous solution of any of claims 1 to 5 and 20.

23. The aqueous solution prepared by the method of claim 22.

24. The aqueous solution of claim 22, which exhibits stability when stored at stress conditions at 50° C. for 4 weeks, when stored at accelerated conditions at 40° C. for 6 months, or when stored at long term conditions.

25. The aqueous solution of claim 24, wherein said stability includes one or more of the following:
a) at least 90% remaining bromfenac;
b) not more than 3.0% total impurity;
c) not more than 0.8% impurity A;
d) not more than 0.8% impurity B.

26. A method of treating ocular inflammation or pain in a patient in need thereof, comprising administering to an eye of the patient an effective amount of an aqueous solution according to claim 25.

27. The bromfenac solution of claim 25, which upon storage for 6 months at accelerated conditions at 40° C. at no more than 25% relative humidity, comprises less than 0.8% of Impurity B {(7-(4-bromobenzoyl) indoline-2,3-dione} of active ingredient.

28. A method of treating ocular inflammation or pain in a patient in need thereof, comprising administering to an eye of the patient an effective amount of a stable pharmaceutical aqueous solution comprising bromfenac, or a pharmaceutically acceptable salt or hydrate thereof; further comprising a polyoxyethylene sorbitan fatty acid ester and/or a polyoxyl-15-hydroxystearate; wherein the solution has a pH of from about 6.5 to about 8; and wherein the solution does not comprise any of an alkyl aryl polyether alcohol type polymer, a polyethylene glycol fatty acid ester, and an antioxidant.

29. Use of a composition for treating ocular inflammation or pain in a patient in need thereof, comprising administering to an eye of the patient an effective amount of an aqueous solution according to claim 25.

30. Use of a composition for treating ocular inflammation or pain in a patient in need thereof, comprising administering to an eye of the patient an effective amount of a stable pharmaceutical aqueous solution comprising bromfenac, or a pharmaceutically acceptable salt or hydrate thereof; further comprising a polyoxyethylene sorbitan fatty acid ester and/or a polyoxyl-15-hydroxystearate; wherein the solution has a pH of from about 6.5 to about 8; and wherein the solution does not comprise any of an alkyl aryl polyether alcohol type polymer, a polyethylene glycol fatty acid ester, and an antioxidant.
